(19) United States
(12) Patent Application Publication
(10) Pub. No.: US 2026/0200912 A1
(43) Pub. Date: Jul. 16, 2026

(54) PROCESSES AND INTERMEDIATES FOR SYNTHESIS OF MRTX0902

(71) Applicant: MIRATI THERAPEUTICS, INC., PRINCETON, NJ (US)

(72) Inventors: THOMAS SCATTOLIN, SAN DIEGO, CA (US); CHENG CHEN, SAN DIEGO, CA (US); MICHAL ACHMATOWICZ, SAN DIEGO, CA (US)

(73) Assignee: MIRATI THERAPEUTICS, INC., PRINCETON, NJ (US)

(21) Appl. No.: 19/133,263

(22) PCT Filed: Nov. 16, 2023
(86) PCT No.: PCT/US2023/080125

§ 371 Date: May 28, 2025

Related U.S. Application Data
(60) Provisional application No. 63/428,658, filed on Nov. 29, 2022.

(51) Int. Cl. C07D 471/04; C07C 57/15

(52) U.S. Cl. CPC C07D 471/04; C07C 57/15

(57) ABSTRACT

The present invention relates to new synthetic routes of manufacturing MRTX0902. The invention also provides intermediates used in the provided synthetic routes, and the fumarate salt of MRTX0902.

### FIELD OF THE INVENTION

The present invention relates to new and improved synthetic routes for synthesis of MRTX0902, as well as to fumaate salts of MRTX0902.

### BACKGROUND OF THE INVENTION

The Ras family comprises v-Ki-ras2 Kirsten rat sarcoma viral oncogene homolog (KRAS), neuroblastoma RAS viral oncogene homolog (NRAS), and Harvey murine sarcoma virus oncogene (HRAS) and critically regulates cellular division, growth and function in normal and altered states including cancer (see e.g., Simanshu et al. Cell, 2017. 170 (1): p. 17-33; Matikas et al., Crit Rev Oncol Hematol, 2017. 110: p. 1-12). RAS proteins are activated by upstream signals, including receptor tyrosine kinases (RTKs), and transduce signals to several downstream signaling pathways such as the mitogen-activated protein kinase (MAPK)/extracellular signal-regulated kinases (ERK) pathway. Hyperactivation of RAS signaling is frequently observed in cancer as a result of mutations or alterations in RAS genes or other genes in the RAS pathway. The identification of strategies to inhibit RAS and RAS signaling are predicted to be useful for the treatment of cancer and RAS-regulated disease states.

RAS proteins are guanosine triphosphatases (GTPases) that cycle between an inactive, guanosine diphosphate (GDP)-bound state and an active guanosine triphosphate (GTP)-bound state. Son of sevenless homolog 1 (SOS1) is a guanine nucleotide exchange factor (GEF) that mediates the exchange of GDP for GTP, thereby activating RAS proteins. RAS proteins hydrolyze GTP to GDP through their intrinsic GTPase activity which is greatly enhanced by GTPase-activating proteins (GAPs). This regulation through GAPs and GEFs is the mechanism whereby activation and deactivation are tightly regulated under normal conditions. Mutations at several residues in all three RAS proteins are frequently observed in cancer and result in RAS remaining predominantly in the activated state (Sanchez-Vega et al., Cell, 2018. 173: p. 321-337 Li et al., Nature Reviews Cancer, 2018. 18: p. 767-777). Mutations at codon 12 and 13 are the most frequently mutated RAS residues and prevent GAP-stimulated GTP hydrolysis by blocking the interaction of GAP proteins and RAS. Recent biochemical analyses however, demonstrated these mutated proteins still require nucleotide cycling for activation based on their intrinsic GTPase activity and/or partial sensitivity to extrinsic GTPases. As such, mutant RAS proteins are sensitive to inhibition of upstream factors such as SOS1 or SHP2, another upstream signaling molecule required for RAS activation (Hillig, 2019; Patricelli, 2016; Lito, 2016; Nichols, 2018).

The three main RAS-GEF families that have been identified in mammalian cells are SOS, RAS-GRF and RAS-GRP (Rojas, 2011). RAS-GRF and RAS-GRP are expressed in the cells of the central nervous system and hematopoietic cells, respectively, while the SOS family is ubiquitously expressed and is responsible for transducing RTK signaling. The SOS family comprises SOS1 and SOS2 and these proteins share approximately 70% sequence identity. SOS1 appears to be much more active than SOS2 due to the rapid degradation of SOS2. The mouse SOS2 knockout is viable whereas the SOS1 knockout is embryonic lethal. A tamoxifen-inducible SOS1 knockout mouse model was used to interrogate the role of SOS1 and SOS2 in adult mice and demonstrated the SOS1 knockout was viable but the SOS1/2 double knockout was not viable (Baltanas, 2013) suggesting functional redundancy and that selective inhibition of SOS1 may have a sufficient therapeutic index for the treatment of SOS1-RAS activated diseases.

SOS proteins are recruited to phosphorylated RTKs through an interaction with growth factor receptor bound protein 2 (GRB2). Recruitment to the plasma membrane places SOS in close proximity to RAS and enables SOS-mediated RAS activation. SOS proteins bind to RAS through a binding site that promotes nucleotide exchange as well as through an allosteric site that binds GTP-bound RAS-family proteins and increases the function of SOS (Freedman et al., Proc. Natl. Acad. Sci, USA 2006. 103 (45): p. 16692-97). Binding to the allosteric site relieves steric occlusion of the RAS substrate binding site and is therefore required for nucleotide exchange. Retention of the active conformation at the catalytic site following interaction with the allosteric site is maintained in isolation due to strengthened interactions of key domains in the activated state. SOS1 mutations are found in Noonan syndrome and several cancers including lung adenocarcinoma, embryonal rhabdomyosarcoma, Sertoli cell testis tumor and granular cell tumors of the skin (see e.g., Denayer, E., et al, Genes Chromosomes Cancer, 2010. 49 (3): p. 242-52).

GTPase-activating proteins (GAPs) are proteins that stimulate the low intrinsic GTPase activity of RAS family members and therefore converts active GTP-bound RAS proteins into inactive, GDP-bound RAS proteins (e.g., see Simanshu, D. K., Cell, 2017, Ras Proteins and their Regulators in Human Disease). While activating alterations in the GEF SOS1 occur in cancers, inactivating mutations and loss-of-function alterations in the GAPs neurofibromin 1 (NF-1) or neurofibromin 2 (NF-2) also occur creating a state where SOS1 activity is unopposed and activity downstream of the pathway through RAS proteins is elevated.

Thus, the compounds of the present invention that block the interaction between SOS1 and Ras-family members prevent the recycling of KRas into the active GTP-bound form and, therefore, may provide therapeutic benefit for a wide range of cancers, particularly Ras family member-associated cancers. The compounds of the present invention offer potential therapeutic benefit as inhibitors of SOS1-KRas interaction that may be useful for negatively modulating the activity of KRas through blocking SOS1-KRas interaction in a cell for treating various forms of cancer, including Ras-associated cancer, SOS1-associated cancer and NF1/NF2-associated cancer.

SOS1 inhibitor compound (R)-2-methyl-3-(1-((4-methyl-7-morpholinopyrido[3,4-d]pyridazin-1-yl)amino)ethyl)benzonitrile (also known as MRTX0902) has the following structure:

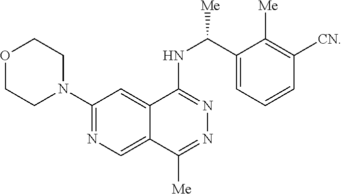 [SMILES: Cc1c(C#N)cccc1[C@@H](C)Nc1nnc(C)c2cnc(N3CCOCC3)cc12]

MRTX0902 is described, for example, in Example 12-10 of PCT Application WO 2021/127429.

While WO 2021/127429 describes methods of making MRTX0902, there is a need in the art for new and improved synthetic routes of making MRTX0902.

### SUMMARY OF THE INVENTION

The present invention, in one embodiment, provides new and improved methods of making MRTX0902 (i.e., (R)-2-methyl-3-(1-((4-methyl-7-morpholinopyrido[3,4-d]pyridazin-1-yl)amino)ethyl)benzonitrile).

In one embodiment, the invention provides a method of synthesizing (R)-2-methyl-3-(1-((4-methyl-7-morpholinopyrido[3,4-d]pyridazin-1-yl)amino)ethyl)benzonitrile, comprising the step of:
a) reacting a compound of the following structure:

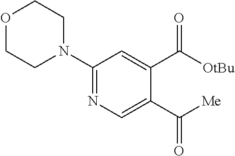 [SMILES: CC(=O)c1cnc(N2CCOCC2)cc1C(=O)OC(C)(C)C]

with hydrazine or a hydrazine salt in the presence of an acid and a solvent to produce a final compound of step a) of the following structure:

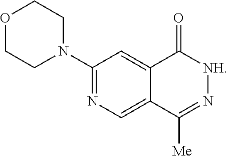 [SMILES: Cc1n[nH]c(=O)c2cc(N3CCOCC3)ncc12]

In one embodiment, step a) is carried out at a temperature from about 30° C. to about 150° C.

In one embodiment, the solvent is selected from the group consisting of dimethylacetamide (DMAc), dimethylformamide (DMF), 1,4-dioxane, tetrahydrofuran (THF), 2-methyltetrahydrofuran (2-MeTHF), acetonitrile (MeCN), dimethyl sulfoxide (DMSO), N-methylpyrrolidone (NMP), toluene and an alcohol with a formula R—OH, wherein R is alkyl, allyl or aryl.

In a preferred embodiment, the solvent is ethanol.

In one embodiment, the acid is selected from the group consisting of a mineral acid and an organic acid.

In one embodiment, the acid is a mineral acid selected from the group consisting of a hydrogen halide of the general formula HX (where X is F, Cl, Br or I), nitric acid, phosphoric acid, sulfuric acid, boric acid and perchloric acid.

In another embodiment, the acid is an organic acid selected from the group consisting of a sulfonic acid of the general formula RSO₃H (where R is alkyl, alkenyl, alkynyl, carbocycle, heterocycle, or aryl), and a carboxylic acid (with one or several carboxylic acid sites) of the general formula RCO₂H (where R is alkyl, alkenyl, alkynyl, carbocycle, heterocycle, or aryl). In one embodiment, the organic acid is selected from the group consisting of lactic acid, acetic acid, formic acid, citric acid, oxalic acid, uric acid, malic acid, and tartaric acid.

In a preferred embodiment, the acid is acetic acid.

In one embodiment, the method further comprises step b):
b) reacting the final compound of step a) with a chlorinating agent in the presence of an aprotic solvent to produce a final compound of step b) of the following structure:

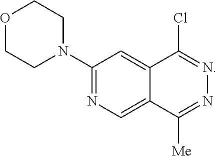 [SMILES: Cc1nnc(Cl)c2cc(N3CCOCC3)ncc12]

In one embodiment, step b) is carried out at a temperature from about 30° C. to about 150° C.

In one embodiment, the chlorinating agent is selected from the group consisting of phosphorus oxychloride, phosphorus trichloride, oxalyl chloride, thionyl chloride, diaryl or dialkyl chlorophosphate, diaryl, and dialkyl chlorophosphite.

In a preferred embodiment, the chlorinating agent is phosphorus oxychloride.

In one embodiment, the aprotic solvent is selected from the group consisting of chloroform, DMAc, DMF, 1,4-dioxane, THF, 2-MeTHF, MeCN, DMSO, toluene, tertiary amine, and NMP.

In a preferred embodiment, the aprotic solvent is MeCN.

In one embodiment, the method further comprises step c):
c) reacting the final compound of step b) with a benzylic amine or a benzylic amine salt in the presence of a base, Lewis acid and a high-boiling solvent to produce a final compound of step c) of the following structure:

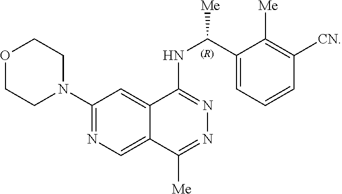

In one embodiment, step c) is carried out at a temperature from about 50° C. to about 170° C.

In one embodiment, the base is selected from the group consisting of an organic base and an inorganic base.

In one embodiment, the base is an organic base selected from the group consisting of DIPEA, Et₃N, DABCO, and DBU.

In a preferred embodiment, the organic base is DIPEA.

In another embodiment, the base is an inorganic base selected from the group consisting of carbonate, bicarbonate, and phosphate (including mono-, di- and tribasic phosphate).

In one embodiment, the Lewis acid is selected from the group consisting of a magnesium salt, a calcium salt, an aluminum-based reagent, and a boron-based reagent.

In a preferred embodiment, the Lewis acid is magnesium chloride.

In one embodiment, the high-boiling solvent is selected from the group consisting of toluene, DMAc, DMF, 1,4-dioxane, DMSO, NMP, and an alcohol with a formula R—OH, wherein R is alkyl, allyl or aryl.

In a preferred embodiment, the high-boiling solvent is tAmOH (tert-Amyl alcohol).

In one embodiment, the invention provides a method of manufacturing (R)-2-methyl-3-(1-((4-methyl-7-morpholinopyrido[3,4-d]pyridazin-1-yl)amino)ethyl)benzonitrile, comprising reacting a compound of the following structure:

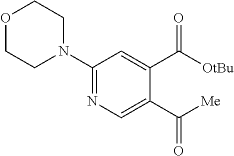 [SMILES: CC(=O)c1cnc(N2CCOCC2)cc1C(=O)OC(C)(C)C]

with hydrazine or a hydrazine salt in the presence of an acid and a solvent to produce a compound of the following structure:

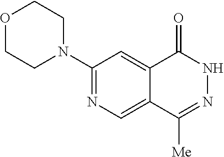 [SMILES: Cc1n[nH]c(=O)c2cc(N3CCOCC3)ncc12]

The preferred embodiments for the solvent, the acid and the temperature range are those as described above for step a).

In another embodiment, the invention provides a method of manufacturing (R)-2-methyl-3-(1-((4-methyl-7-morpholinopyrido[3,4-d]pyridazin-1-yl)amino)ethyl)benzonitrile, comprising:
reacting

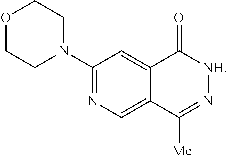 [SMILES: Cc1n[nH]c(=O)c2cc(N3CCOCC3)ncc12]

with a chlorinating agent in the presence of an aprotic solvent to produce a compound of the following structure:

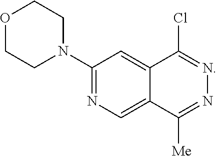 [SMILES: Cc1nnc(Cl)c2cc(N3CCOCC3)ncc12]

The preferred embodiments for the chlorinating agent, the aprotic solvent and the temperature range are those as described above for step b).

In another embodiment, the invention provides a method of manufacturing MRTX0902, comprising:
reacting

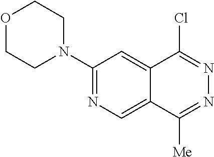 [SMILES: Cc1nnc(Cl)c2cc(N3CCOCC3)ncc12]

with a benzylic amine or a benzylic amine salt in the presence of a base, Lewis acid and a high-boiling solvent to produce a compound with the following structure:

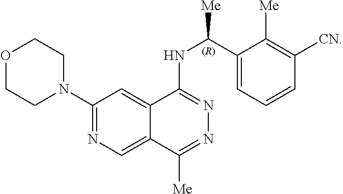

The preferred embodiments for the base, the Lewis acid, the high-boiling solvent and the temperature range are those as described above for step c).

In another embodiment, the invention provides a method of manufacturing (R)-2-methyl-3-(1-((4-methyl-7-morpholinopyrido[3,4-d]pyridazin-1-yl)amino)ethyl)benzonitrile, comprising:
reacting a compound of the following structure

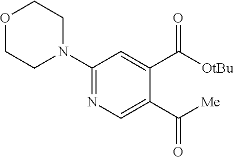 [SMILES: CC(=O)c1cnc(N2CCOCC2)cc1C(=O)OC(C)(C)C]

with hydrazine or a hydrazine salt in the presence of an acid and a solvent to produce a compound of the following structure:

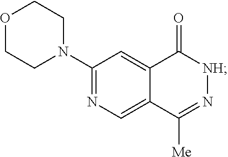 [SMILES: Cc1n[nH]c(=O)c2cc(N3CCOCC3)ncc12]

reacting

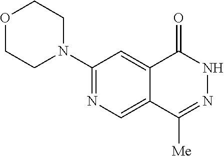 [SMILES: Cc1n[nH]c(=O)c2cc(N3CCOCC3)ncc12]

with a chlorinating agent in the presence of an aprotic solvent to produce a compound of the following structure:

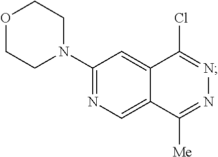 [SMILES: Cc1nnc(Cl)c2cc(N3CCOCC3)ncc12]

reacting

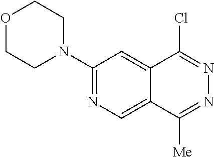 [SMILES: Cc1nnc(Cl)c2cc(N3CCOCC3)ncc12]

with a benzylic amine in the presence of a base, Lewis acid and a high-boiling solvent to produce a compound with the following structure:

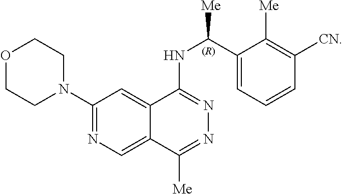

The preferred embodiments for the solvent, the acid, the chlorinating agent, the aprotic solvent, the base, the Lewis acid, the high-boiling solvent and the temperature ranges are those as described above for steps a), b) and c).

In one embodiment, the invention provides a method of manufacturing (R)-2-methyl-3-(1-((4-methyl-7-morpholinopyrido[3,4-d]pyridazin-1-yl)amino)ethyl)benzonitrile, comprising
reacting a compound of the following structure:

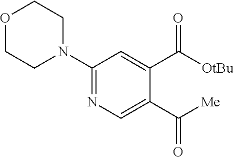 [SMILES: CC(=O)c1cnc(N2CCOCC2)cc1C(=O)OC(C)(C)C]

with hydrazine hydrate in the presence of acetic acid and ethanol to produce a compound with the following structure:

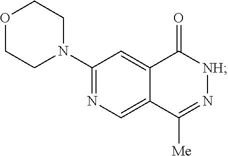 [SMILES: Cc1n[nH]c(=O)c2cc(N3CCOCC3)ncc12]

reacting

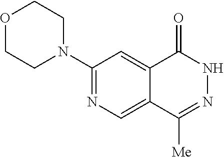 [SMILES: Cc1n[nH]c(=O)c2cc(N3CCOCC3)ncc12]

with phosphorus oxychloride in the presence of MeCN to produce a compound of the following structure:

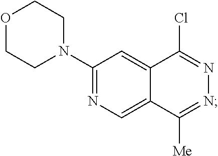 [SMILES: Cc1nnc(Cl)c2cc(N3CCOCC3)ncc12]

reacting

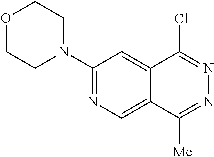 [SMILES: Cc1nnc(Cl)c2cc(N3CCOCC3)ncc12]

with

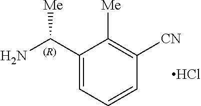

in the presence of DIPEA, MgCl₂, and tAmOH to produce a compound with the following structure:

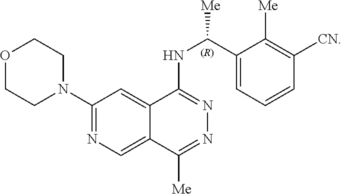

In another embodiment, the invention provides a method of manufacturing a fumarate salt of (R)-2-methyl-3-(1-((4-methyl-7-morpholinopyrido[3,4-d]pyridazin-1-yl)amino)ethyl)benzonitrile, comprising
reacting a compound of the following structure:

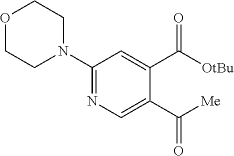 [SMILES: CC(=O)c1cnc(N2CCOCC2)cc1C(=O)OC(C)(C)C]

with hydrazine or a hydrazine salt in the presence of an acid and a solvent to produce a compound with the following structure:

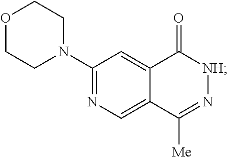 [SMILES: Cc1n[nH]c(=O)c2cc(N3CCOCC3)ncc12]

reacting

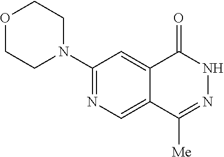 [SMILES: Cc1n[nH]c(=O)c2cc(N3CCOCC3)ncc12]

with a chlorinating agent in the presence of an aprotic solvent to produce a compound of the following structure:

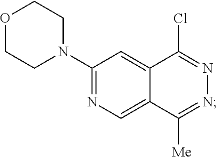 [SMILES: Cc1nnc(Cl)c2cc(N3CCOCC3)ncc12]

reacting

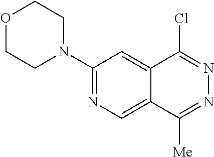 [SMILES: Cc1nnc(Cl)c2cc(N3CCOCC3)ncc12]

with a benzylic amine in the presence of a base, Lewis acid and a high-boiling solvent to produce a compound with the following structure:

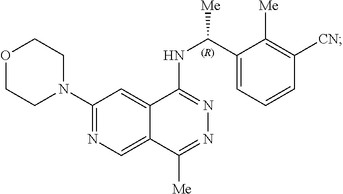

and
reacting

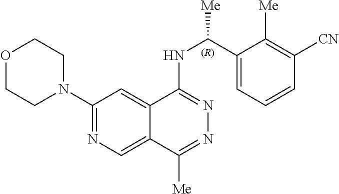

with fumaric acid in the presence of a solvent to produce a compound with the following structure:

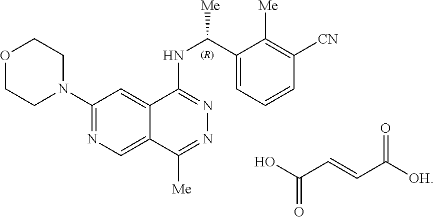

The preferred embodiments for the solvent, the acid, the chlorinating agent, the aprotic solvent, the base, the Lewis acid, the high-boiling solvent and the temperature ranges used in the first three steps are those as described above for steps a), b) and c).

In one embodiment, in the fourth step (reacting (R)-2-methyl-3-(1-((4-methyl-7-morpholinopyrido[3,4-d]pyridazin-1-yl)amino)ethyl)benzonitrile with fumaric acid), the solvent is selected from the group consisting of DMAc, DMF, THF, 2-MeTHF, MeCN, DMSO, NMP, toluene and an alcohol with a formula R—OH, wherein R is alkyl, allyl or aryl. In a preferred embodiment, the solvent is ethanol.

In a preferred embodiment, the solvent is ethanol.

In one embodiment, the fourth step is carried out at a temperature from about 15° C. to about 135° C.

In another embodiment, the invention provides a method of manufacturing a fumarate salt of (R)-2-methyl-3-(1-((4-methyl-7-morpholinopyrido[3,4-d]pyridazin-1-yl)amino)ethyl)benzonitrile, comprising
reacting a compound of the following structure:

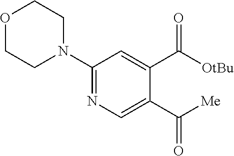 [SMILES: CC(=O)c1cnc(N2CCOCC2)cc1C(=O)OC(C)(C)C]

with hydrazine hydrate in the presence of acetic acid and ethanol to produce a compound with the following structure:

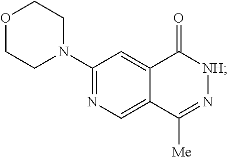 [SMILES: Cc1n[nH]c(=O)c2cc(N3CCOCC3)ncc12]

reacting

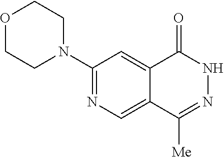 [SMILES: Cc1n[nH]c(=O)c2cc(N3CCOCC3)ncc12]

with phosphorus oxychloride in the presence of MeCN to produce a compound of the following structure:

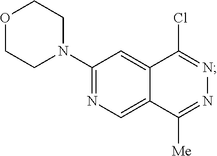 [SMILES: Cc1nnc(Cl)c2cc(N3CCOCC3)ncc12]

reacting

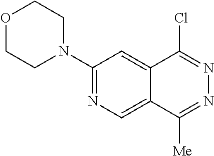 [SMILES: Cc1nnc(Cl)c2cc(N3CCOCC3)ncc12]

with

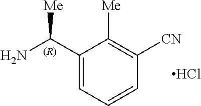

in the presence of DIPEA, MgCl₂, and tAmOH to produce a compound with the following structure:

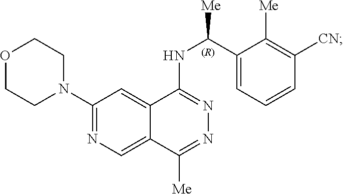

and
reacting

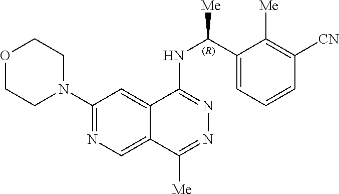

with fumaric acid in the presence of ethanol to produce a compound with the following structure:

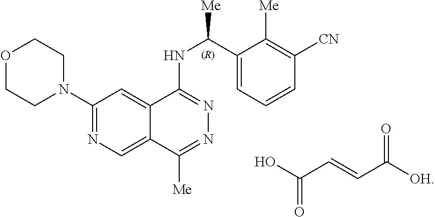

In another embodiment, the invention provides novel intermediate compounds, such as:

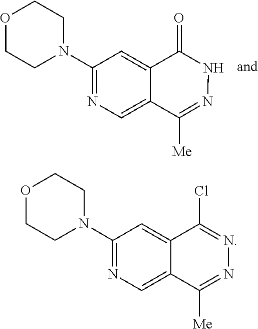

In another embodiment, the invention provides a fumarate salt of MRTX0902 with the following structure:

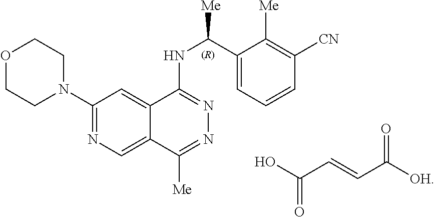

### DETAILED DESCRIPTION OF THE INVENTION

The present invention relates to new synthetic routes for synthesizing MRTX0902, as well as to novel intermediates used in the provided routes, and to fumarate salt of MRTX0902.

Although there is a known method of synthesizing MRTX0902 (see WO 2021/127429), the synthesis provided by the present invention is much improved, in that it has fewer steps, provides a much higher isolated yield and a higher purity overall.

In addition, the expensive chiral benzylic amine is now introduced toward the end of the synthesis, thus maximizing its usage. The sequence of reactions was also modified to increase the yield and the selectivity, automatically reducing associated cost, and drastically improving the purity, while avoiding the use of tedious purification methods.

The overall yield of MRTX0902 increased almost four-fold (18% to 69%) using the described process and was demonstrated on multi-kilo scale (8 kg) with a shorter time cycle.

### Definitions

Unless defined otherwise, all technical and scientific terms used herein have the same meaning as is commonly understood by one of skill in the art to which this invention belongs. All patents, patent applications, and publications referred to herein are incorporated by reference.

As used herein, “KRas G12C” refers to a mutant form of a mammalian KRas protein that contains an amino acid substitution of a cysteine for a glycine at amino acid position 12. The assignment of amino acid codon and residue positions for human KRas is based on the amino acid sequence identified by UniProtKB/Swiss-Prot P01116: Variant p.Glyl2Cys.

A “KRas G12C-associated disease or disorder” as used herein refers to diseases or disorders associated with or mediated by or having a KRas G12C mutation. A non-limiting example of a KRas G12C-associated disease or disorder is a KRas G12C-associated cancer.

As used herein, the term “MRTX0902” refers to the compound which has the name (R)-2-methyl-3-(1-((4-methyl-7-morpholinopyrido[3,4-d]pyridazin-1-yl)amino)ethyl)benzonitrile and has the following structure:

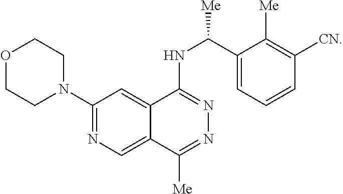 [SMILES: Cc1c(C#N)cccc1[C@@H](C)Nc1nnc(C)c2cnc(N3CCOCC3)cc12]

MRTX0902 is described, for example, in Example 12-10 of PCT Application WO 2021/127429.

The term “MRTX0902” encompasses all chiral (enantiomeric and diastereomeric) and racemic forms of the compound.

In one embodiment, the term “MRTX0902” includes salts of the above compound, for instance salts formed with inorganic acids such as hydrochloric acid, hydrobromic acid, sulfuric acid, phosphoric acid, nitric acid, salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, polygalacturonic acid, and fumaric acid, and salts formed from quaternary ammoniums of the formula —NR+Z—, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, cinnamoate, mandeloate, benzyloate, and diphenylacetate).

As used herein, “SOS1” refers to a mammalian Son of sevenless homolog 1 (SOS1) enzyme.

A “SOS1-associated disease or disorder” as used herein refers to diseases or disorders associated with or mediated by or having an activating SOS1 mutation. Examples of activating SOS1 mutations include SOS1 N233S and SOS1 N233Y mutations.

A “SOS1-associated disease or disorder” as used herein refers to diseases or disorders associated with or mediated by or having an activating SOS1 mutation. Examples of activating SOS1 mutations include SOS1 N233S and SOS1 N233Y mutations.

As used herein, “SOS1 N233S” refers to a mutant form of a mammalian SOS1 protein that contains an amino acid substitution of a serine for a glutamine at amino acid position 233. The assignment of amino acid codon and residue positions for human SOS1 is based on the amino acid sequence identified by UniProtKB/Swiss-Prot Q07889: Variant p.Gln233Ser.

As used herein, “SOS1 N233Y” refers to a mutant form of a mammalian SOS1 protein that contains an amino acid substitution of a tyrosine for a glutamine at amino acid position 233. The assignment of amino acid codon and residue positions for human SOS1 is based on the amino acid sequence identified by UniProtKB/Swiss-Prot Q07889: Variant p.Gln233Tyr.

Whenever the application refers to a chemical compound, unless specifically stated otherwise, the compound encompasses all chiral (enantiomeric and diastereomeric) and racemic forms of the compound.

Unless the application specifies differently, “R” refers to a group such as alkyl, alkenyl, alkynyl, alkylene, alkenylene, alkynylene, carbocycle, cycloalkyl, heteroalkyl, heterocycle, aryl, aralkyl, or arylalkyl.

The term “alkyl” is intended to mean a straight chain or branched aliphatic group having from 1 to 12 carbon atoms, alternatively 1-8 carbon atoms, and alternatively 1-6 carbon atoms. Other examples of alkyl groups have from 2 to 12 carbon atoms, alternatively 2-8 carbon atoms and alternatively 2-6 carbon atoms. Examples of alkyl groups include, without limitation, methyl, ethyl, propyl, isopropyl, butyl, isobutyl, sec-butyl, tert-butyl, pentyl, hexyl and the like. A “C0” alkyl (as in “C0-C3alkyl”) is a covalent bond.

The term “alkenyl” is intended to mean an unsaturated straight chain or branched aliphatic group with one or more carbon-carbon double bonds, having from 2 to 12 carbon atoms, alternatively 2-8 carbon atoms, and alternatively 2-6 carbon atoms. Examples of alkenyl groups include, without limitation, ethenyl, propenyl, butenyl, pentenyl, and hexenyl.

The term “alkynyl” is intended to mean an unsaturated straight chain or branched aliphatic group with one or more carbon-carbon triple bonds, having from 2 to 12 carbon atoms, alternatively 2-8 carbon atoms, and alternatively 2-6 carbon atoms. Examples of alkynyl groups include, without limitation, ethynyl, propynyl, butynyl, pentynyl, and hexynyl.

The terms “alkylene,” “alkenylene,” or “alkynylene” as used herein are intended to mean an alkyl, alkenyl, or alkynyl group, respectively, as defined hereinabove, that is positioned between and serves to connect two other chemical groups. Examples of alkylene groups include, without limitation, methylene, ethylene, propylene, and butylene. Examples of alkenylene groups include, without limitation, ethenylene, propenylene, and butenylene. Examples of alkynylene groups include, without limitation, ethynylene, propynylene, and butynylene.

The term “carbocycle” as employed herein is intended to mean a cycloalkyl or aryl moiety.

The term “cycloalkyl” is intended to mean a saturated or unsaturated mono-, bi-, tri- or poly-cyclic hydrocarbon group having about 3 to 15 carbons, alternatively having 3 to 12 carbons, alternatively 3 to 8 carbons, alternatively 3 to 6 carbons, and alternatively 5 or 6 carbons. In certain embodiments, the cycloalkyl group is fused to an aryl, heteroaryl or heterocyclic group. Examples of cycloalkyl groups include, without limitation, cyclopenten-2-enone, cyclopenten-2-enol, cyclohex-2-enone, cyclohex-2-enol, cyclopropyl, cyclobutyl, cyclobutenyl, cyclopentyl, cyclopentenyl, cyclohexyl, cyclohexenyl, cycloheptyl, cyclooctyl, etc.

The term “heteroalkyl” is intended to mean a saturated or unsaturated, straight chain or branched aliphatic group, wherein one or more carbon atoms in the group are independently replaced by a heteroatom selected from the group consisting of O, S, and N.

The term “aryl” is intended to mean a mono-, bi-, tri- or polycyclic aromatic moiety, for example a C6-C14aromatic moiety, for example comprising one to three aromatic rings. Alternatively, the aryl group is a C6-C10aryl group, alternatively a C6aryl group. Examples of aryl groups include, without limitation, phenyl, naphthyl, anthracenyl, and fluorenyl.

The terms “aralkyl” or “arylalkyl” are intended to mean a group comprising an aryl group covalently linked to an alkyl group. If an aralkyl group is described as “optionally substituted”, it is intended that either or both of the aryl and alkyl moieties may independently be optionally substituted or unsubstituted. Alternatively, the aralkyl group is (C1-C6)alk (C6-C10) aryl, including, without limitation, benzyl, phenethyl, and naphthylmethyl. For simplicity, when written as “arylalkyl” this term, and terms related thereto, is intended to indicate the order of groups in a compound as “aryl-alkyl”. Similarly, “alkyl-aryl” is intended to indicate the order of the groups in a compound as “alkyl-aryl”.

As used herein, the term “pharmaceutically acceptable salt” refers to salts that retain the desired biological activity of the above-identified compounds and exhibit minimal or no undesired toxicological effects. Examples of such salts include, but are not limited to acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid. The compounds can also be administered as pharmaceutically acceptable quaternary salts known by those skilled in the art, which specifically include the quaternary ammonium salt of the formula —NR+Z-, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

As used herein, the term “mineral acid” (or “inorganic acid”) refers to any acid derived from an inorganic compound that dissociates to produce hydrogen ions (H+) in water. Nonlimiting examples of mineral acids include hydrogen halides of the general formula HX (where X is F, Cl, Br or I), nitric acid, phosphoric acid, sulfuric acid, boric acid and perchloric acid.

As used herein, the term “organic acid” refers to any organic compound with acidic properties. Nonlimiting examples of organic acids include sulfonic acids of the general formula RSO₃H (where R can be alkyl, alkenyl, alkynyl, carbocycle, heterocycle, aryl and are define above), and carboxylic acids (with one or several carboxylic acid sites) of the general formula RCO₂H (where R can be alkyl, alkenyl, alkynyl, carbocycle, heterocycle, aryl and are define above). Nonlimiting examples of organic acids are lactic acid, acetic acid, formic acid, citric acid, oxalic acid, uric acid, malic acid, and tartaric acid.

### Synthetic Schemes

In one embodiment, the invention provides a method of synthesizing MRTX0902, comprising the step of:
a) reacting a compound of the following structure:

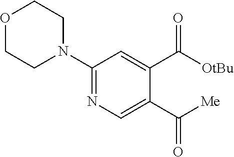 [SMILES: CC(=O)c1cnc(N2CCOCC2)cc1C(=O)OC(C)(C)C]

with hydrazine or a hydrazine salt in the presence of an acid and a solvent to produce a final compound of step a) of the following structure:

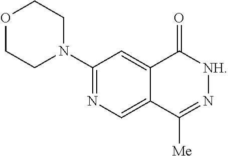 [SMILES: Cc1n[nH]c(=O)c2cc(N3CCOCC3)ncc12]

In one embodiment, step a) is carried out at a temperature from about 30° C. to about 150° C.

In one embodiment, the solvent is selected from the group consisting of dimethylacetamide (DMAc), dimethylformamide (DMF), 1,4-dioxane, tetrahydrofuran (THF), 2-methyltetrahydrofuran (2-MeTHF), acetonitrile (MeCN), dimethyl sulfoxide (DMSO), N-methylpyrrolidone (NMP), toluene and an alcohol with a formula R—OH, wherein R is alkyl, allyl or aryl.

In one embodiment, the solvent comprises, but is not limited to, one or more of dimethylacetamide (DMAc), dimethylformamide (DMF), 1,4-dioxane, tetrahydrofuran (THF), 2-methyltetrahydrofuran (2-MeTHF), acetonitrile (MeCN), dimethyl sulfoxide (DMSO), N-methylpyrrolidone (NMP), toluene and an alcohol with a formula R—OH, wherein R is alkyl, allyl or aryl.

In a preferred embodiment, the solvent is ethanol.

In one embodiment, the acid is selected from the group consisting of a mineral acid and an organic acid.

In one embodiment, the acid is a mineral acid selected from the group consisting of a hydrogen halide of the general formula HX (where X is F, Cl, Br or I), nitric acid, phosphoric acid, sulfuric acid, boric acid and perchloric acid.

In one embodiment, the acid is a mineral acid that comprises, but is not limited to, one or more of hydrogen halide of the general formula HX (where X is F, Cl, Br or I), nitric acid, phosphoric acid, sulfuric acid, boric acid and perchloric acid.

In another embodiment, the acid is an organic acid selected from the group consisting of a sulfonic acid of the general formula RSO₃H (where R is alkyl, alkenyl, alkynyl, carbocycle, heterocycle, or aryl), and carboxylic acid (with one or several carboxylic acid sites) of the general formula RCO₂H (where R is alkyl, alkenyl, alkynyl, carbocycle, heterocycle, or aryl). In one embodiment, the organic acid is selected from the group consisting of lactic acid, acetic acid, formic acid, citric acid, oxalic acid, uric acid, malic acid, and tartaric acid.

In one embodiment, the acid is an organic acid that comprises, but is not limited to, one or more of a sulfonic acid of the general formula RSO₃H (where R is alkyl, alkenyl, alkynyl, carbocycle, heterocycle, or aryl), and carboxylic acid (with one or several carboxylic acid sites) of the general formula RCO₂H (where R is alkyl, alkenyl, alkynyl, carbocycle, heterocycle, or aryl).

In a preferred embodiment, the acid is acetic acid.

In one embodiment, the method further comprises step b):
b) reacting the final compound of step a) with a chlorinating agent in the presence of an aprotic solvent to produce a final compound of step b) of the following structure:

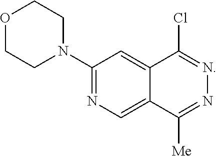 [SMILES: Cc1nnc(Cl)c2cc(N3CCOCC3)ncc12]

In one embodiment, step b) is carried out at a temperature from about 30° C. to about 150° C.

In one embodiment, the chlorinating agent is selected from the group consisting of phosphorus oxychloride, phosphorus trichloride, oxalyl chloride, thionyl chloride, diaryl or dialkyl chlorophosphate, diaryl, and dialkyl chlorophosphite.

In one embodiment, the chlorinating agent comprises, but is not limited to, one or more of phosphorus oxychloride, phosphorus trichloride, oxalyl chloride, thionyl chloride, diaryl or dialkyl chlorophosphate, diaryl, and dialkyl chlorophosphite.

In a preferred embodiment, the chlorinating agent is phosphorus oxychloride.

In one embodiment, the aprotic solvent is selected from the group consisting of chloroform, DMAc, DMF, 1,4-dioxane, THF, 2-MeTHF, MeCN, DMSO, toluene, tertiary amine, and NMP.

In one embodiment, the aprotic solvent comprises, but is not limited to, one or more of chloroform, DMAc, DMF, 1,4-dioxane, THF, 2-MeTHF, MeCN, DMSO, toluene, tertiary amine, and NMP.

In a preferred embodiment, the aprotic solvent is MeCN.

In one embodiment, the method further comprises step c):
c) reacting the final compound of step b) with a benzylic amine or a benzylic amine salt in the presence of a base, Lewis acid and a high-boiling solvent to produce a final compound of step c) of the following structure:

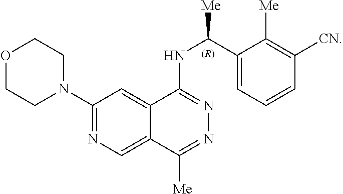

In one embodiment, step c) is carried out at a temperature from about 50° C. to about 170° C.

In one embodiment, the base is selected from the group consisting of an organic base and an inorganic base.

In one embodiment, the base is an organic base selected from the group consisting of DIPEA, Et₃N, DABCO, and DBU.

In one embodiment, the base is an organic base that comprises, but is not limited to, one or more of DIPEA, Et₃N, DABCO, and DBU.

In a preferred embodiment, the organic base is DIPEA.

In another embodiment, the base is an inorganic base selected from the group consisting of carbonate, bicarbonate, and phosphate (including mono-, di- and tribasic phosphate).

In another embodiment, the base is an inorganic base that comprises, but is not limited to, one or more of carbonate, bicarbonate, and phosphate (including mono-, di- and tribasic phosphate).

In one embodiment, the Lewis acid is selected from the group consisting of a magnesium salt, a calcium salt, an aluminum-based reagent, and a boron-based reagent.

In one embodiment, the Lewis acid comprises, but is not limited to, one or more of a magnesium salt, a calcium salt, an aluminum-based reagent, and a boron-based reagent.

In a preferred embodiment, the Lewis acid is magnesium chloride.

In one embodiment, the high-boiling solvent is selected from the group consisting of toluene, DMAc, DMF, 1,4-dioxane, DMSO, NMP, and an alcohol with a formula R—OH, wherein R is alkyl, allyl or aryl.

In one embodiment, the high-boiling solvent comprises, but is not limited to, one or more of toluene, DMAc, DMF, 1,4-dioxane, DMSO, NMP, and an alcohol with a formula R—OH, wherein R is alkyl, allyl or aryl.

In a preferred embodiment, the high-boiling solvent is tAmOH (tert-Amyl alcohol).

In one embodiment, the invention provides a method of manufacturing MRTX0902, comprising reacting a compound of the following structure:

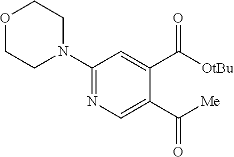 [SMILES: CC(=O)c1cnc(N2CCOCC2)cc1C(=O)OC(C)(C)C]

with hydrazine or a hydrazine salt in the presence of an acid and a solvent to produce a compound of the following structure:

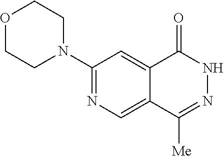 [SMILES: Cc1n[nH]c(=O)c2cc(N3CCOCC3)ncc12]

The preferred embodiments for the solvent, the acid and the temperature range are those as described above for step a).

In another embodiment, the invention provides a method of manufacturing MRTX0902, comprising reacting

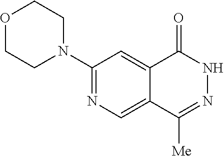 [SMILES: Cc1n[nH]c(=O)c2cc(N3CCOCC3)ncc12]

with a chlorinating agent in the presence of an aprotic solvent to produce a compound of the following structure:

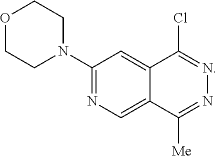 [SMILES: Cc1nnc(Cl)c2cc(N3CCOCC3)ncc12]

The preferred embodiments for the chlorinating agent, the aprotic solvent and the temperature range are those as described above for step b).

In another embodiment, the invention provides a method of manufacturing MRTX0902, comprising:

reacting

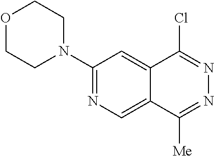 [SMILES: Cc1nnc(Cl)c2cc(N3CCOCC3)ncc12]

with a benzylic amine or a benzylic amine salt in the presence of a base, Lewis acid and a high-boiling solvent to produce a compound with the following structure:

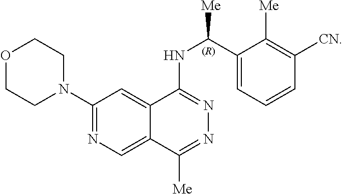

The preferred embodiments for the base, the Lewis acid, the high-boiling solvent and the temperature range are those as described above for step c).

In another embodiment, the invention provides a method of manufacturing MRTX0902, comprising:
reacting a compound of the following structure

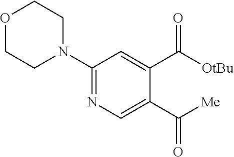 [SMILES: CC(=O)c1cnc(N2CCOCC2)cc1C(=O)OC(C)(C)C]

with hydrazine or a hydrazine salt in the presence of an acid and a solvent to produce a compound of the following structure:

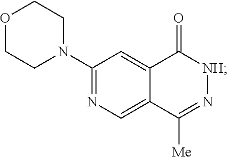 [SMILES: Cc1n[nH]c(=O)c2cc(N3CCOCC3)ncc12]

reacting

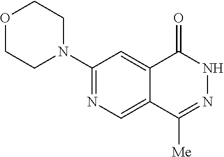 [SMILES: Cc1n[nH]c(=O)c2cc(N3CCOCC3)ncc12]

with a chlorinating agent in the presence of an aprotic solvent to produce a compound of the following structure:

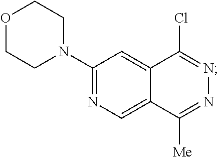 [SMILES: Cc1nnc(Cl)c2cc(N3CCOCC3)ncc12]

reacting

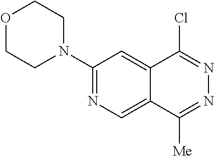 [SMILES: Cc1nnc(Cl)c2cc(N3CCOCC3)ncc12]

with a benzylic amine or a benzylic amine salt in the presence of a base, Lewis acid and a high-boiling solvent to produce a compound with the following structure:

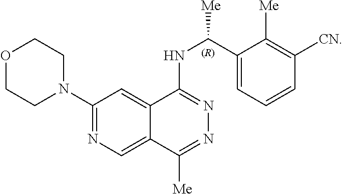

The preferred embodiments for the solvent, the acid, the chlorinating agent, the aprotic solvent, the base, the Lewis acid, the high-boiling solvent and the temperature ranges are those as described above for steps a), b) and c).

In one embodiment, the invention provides a method of manufacturing MRTX0902, comprising
reacting a compound of the following structure:

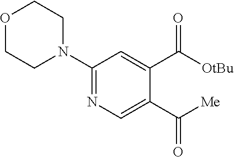 [SMILES: CC(=O)c1cnc(N2CCOCC2)cc1C(=O)OC(C)(C)C]

with hydrazine hydrate in the presence of acetic acid and ethanol to produce a compound with the following structure:

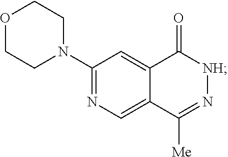 [SMILES: Cc1n[nH]c(=O)c2cc(N3CCOCC3)ncc12]

reacting

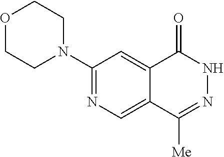 [SMILES: Cc1n[nH]c(=O)c2cc(N3CCOCC3)ncc12]

with phosphorus oxychloride in the presence of MeCN to produce a compound of the following structure:

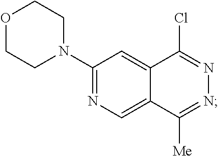 [SMILES: Cc1nnc(Cl)c2cc(N3CCOCC3)ncc12]

reacting

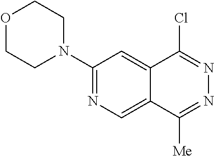 [SMILES: Cc1nnc(Cl)c2cc(N3CCOCC3)ncc12]

with

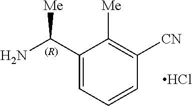

in the presence of DIPEA, MgCl₂, and tAmOH to produce a compound with the following structure:

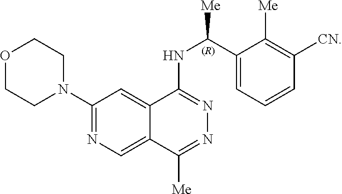

In another embodiment, the invention provides a method of manufacturing a fumarate salt of MRTX0902, comprising
reacting a compound of the following structure:

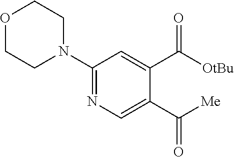 [SMILES: CC(=O)c1cnc(N2CCOCC2)cc1C(=O)OC(C)(C)C]

with hydrazine or a hydrazine salt in the presence of an acid and a solvent to produce a compound with the following structure:

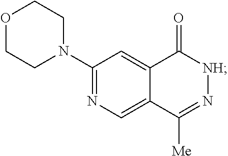 [SMILES: Cc1n[nH]c(=O)c2cc(N3CCOCC3)ncc12]

reacting

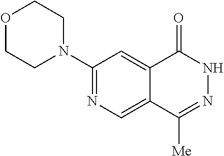 [SMILES: Cc1n[nH]c(=O)c2cc(N3CCOCC3)ncc12]

with a chlorinating agent in the presence of an aprotic solvent to produce a compound of the following structure:

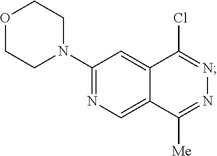 [SMILES: Cc1nnc(Cl)c2cc(N3CCOCC3)ncc12]

reacting

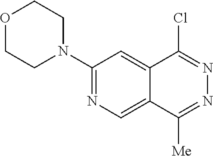 [SMILES: Cc1nnc(Cl)c2cc(N3CCOCC3)ncc12]

with a benzylic amine or a benzylic amine salt in the presence of a base, Lewis acid and a high-boiling solvent to produce a compound with the following structure:

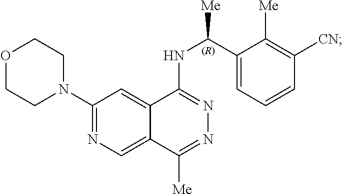

and
reacting

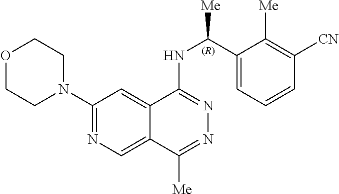

with fumaric acid in the presence of a solvent to produce a compound with the following structure:

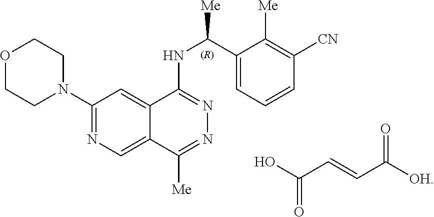

The preferred embodiments for the solvent, the acid, the chlorinating agent, the aprotic solvent, the base, the Lewis acid, the high-boiling solvent and the temperature ranges used in the first three steps are those as described above for steps a), b) and c).

In one embodiment, in the fourth step (reacting MRTX0902 with fumaric acid), the solvent is selected from the group consisting of DMAc, DMF, THE, 2-MeTHF, MeCN, DMSO, NMP, toluene and an alcohol with a formula R—OH, wherein R is alkyl, allyl or aryl. In a preferred embodiment, the solvent is ethanol.

In one embodiment, in the fourth step (reacting MRTX0902 with fumaric acid), the solvent comprises, but is not limited to, one or more of DMAc, DMF, THF, 2-MeTHF, MeCN, DMSO, NMP, toluene and an alcohol with a formula R—OH, wherein R is alkyl, allyl or aryl.

In a preferred embodiment, the solvent is ethanol.

In one embodiment, the fourth step is carried out at a temperature from about 15° C. to about 135° C.

In another embodiment, the invention provides a method of manufacturing a fumarate salt of MRTX0902, comprising
reacting a compound of the following structure:

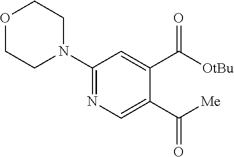 [SMILES: CC(=O)c1cnc(N2CCOCC2)cc1C(=O)OC(C)(C)C]

with hydrazine hydrate in the presence of acetic acid and ethanol to produce a compound with the following structure:

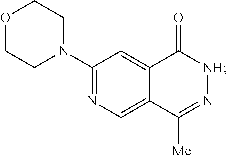 [SMILES: Cc1n[nH]c(=O)c2cc(N3CCOCC3)ncc12]

reacting

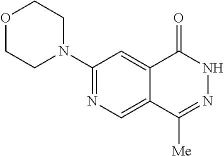 [SMILES: Cc1n[nH]c(=O)c2cc(N3CCOCC3)ncc12]

with phosphorus oxychloride in the presence of MeCN to produce a compound of the following structure:

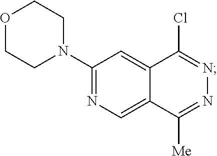 [SMILES: Cc1nnc(Cl)c2cc(N3CCOCC3)ncc12]

reacting

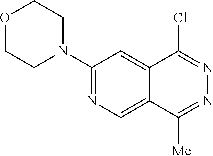 [SMILES: Cc1nnc(Cl)c2cc(N3CCOCC3)ncc12]

with

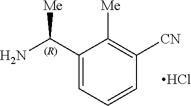

in the presence of DIPEA, MgCl₂, and tAmOH to produce a compound with the following structure:

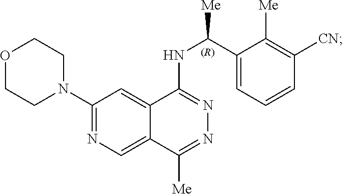

and
reacting

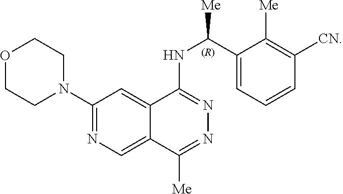

with fumaric acid in the presence of ethanol to produce a compound with the following structure:

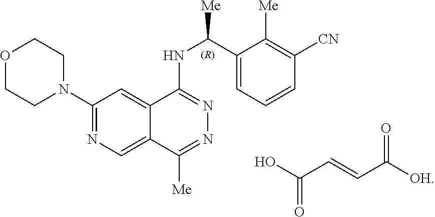

In another embodiment, the invention provides novel intermediate compounds, such as:

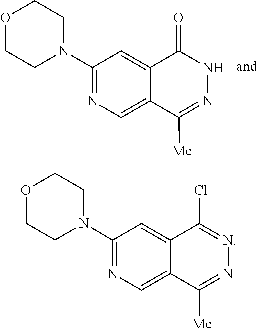

In another embodiment, the invention provides a fumarate salt of MRTX0902 with the following structure:

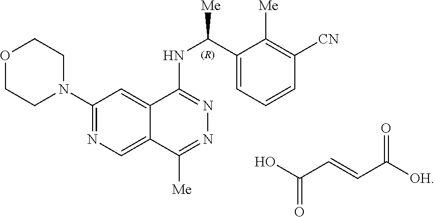

The following Examples are intended to illustrate further certain embodiments of the invention and are not intended to limit the scope of the invention.

### Example 1

### Step (a)

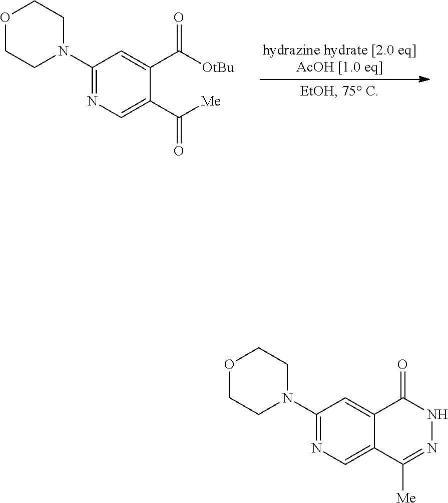 [SMILES: CC(=O)c1cnc(N2CCOCC2)cc1C(=O)OC(C)(C)C ; Cc1n[nH]c(=O)c2cc(N3CCOCC3)ncc12]

To a 100 L reactor was added tert-butyl 5-acetyl-2-morpholinoisonicotinate [7.90 kg, 25.8 mol, 1.0 equiv.] followed by EtOH [55.3 L]. Then acetic acid [1.58 kg, 25.8 mol, 1.0 equiv.] was added to the stirred suspension at 20° C. Hydrazine hydrate 80% [3.2 kg, 51.6 mol, 2.0 equiv.] was added slowly while the temperature was controlled at NMT 35° C. At the end of the addition the temperature was increased to 75° C. The mixture was allowed to react at 75° C. until the starting material was ≤1.0 area % by HPLC analysis (typically 5-10 h). The reaction was cooled to 20° C. The precipitated solid was filtered, and the cake was washed with EtOH (7.9 L×2). The wet cake was then dried at 50° C. under vacuum to afford 5.96 kg of 4-methyl-7-morpholinopyrido[3,4-d]pyridazin-1 (2H)-one in 94% yield.

M.p.: 249.0° C. (dec.).

¹H NMR (400 MHz, DMSO-d₆) δ 12.25 (s, 1H), 8.89 (s, 1H), 7.23 (s, 1H), 3.74-3.72 (m, 4H), 3.67-3.67 (m, 4H), 2.47 (s, 3H).

¹³C NMR (101 MHz, DMSO-d₆) δ 156.0, 159.2, 149.4, 143.2, 135.2, 116.2, 98.4, 66.3, 45.2, 18.1.

HRMS (ESI) calculated for C₁₂H₁₅N₄O₂:247.1190 [M+H]⁺, Found: 247.1190.

### Example 2

### Step (b)

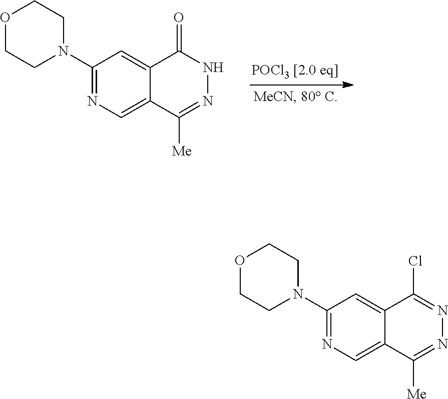 [SMILES: Cc1n[nH]c(=O)c2cc(N3CCOCC3)ncc12 ; Cc1nnc(Cl)c2cc(N3CCOCC3)ncc12]

To a 100 L reactor was added 4-methyl-7-morpholinopyrido[3,4-d]pyridazin-1 (2H)-one [5.93 kg, 24.1 mol, 1.0 equiv.] followed by MeCN [59.3 L]. Then phosphorus oxychloride [7.40 kg, 48.2 mol, 2.0 equiv.] was added at the controlled temperature 15 to 30° C. At the end of the addition the temperature was increased to 75° C. The mixture was allowed to react at 80° C. until the starting material was ≤1.0 area % by HPLC analysis (typically 4-7 h). The reaction was cooled to 20° C. and volatiles were removed under reduced pressure then CH₂Cl₂ [29.5 L] was added. Water [56.9 L] was added followed by sodium carbonate to reach pH=7 to 8. The aqueous phase was discarded, and volatiles were removed under reduced pressure. Then iPrOH was added [26.7 L] and heated to 50° C. for 2 h. Reaction was cooled to 20° C. over 3 h. The precipitated solid was filtered, and the cake was washed once with 2-propanol [5.9 L]. The wet cake was then dried at 50° C. under vacuum to afford 6.12 kg of 4-(1-chloro-4-methylpyrido[3,4-d]pyridazin-7-yl) morpholine in 96% yield.

M.p.: 184.1-184.2° C.

¹H NMR (400 MHz, DMSO-d₆) δ 9.29 (s, 1H), 6.90 (s, 1H), 3.74 (m, 8H), 2.82 (s, 3H).

¹³C NMR (101 MHz, DMSO-d₆) δ 160.1, 157.5, 152.6, 151.7, 131.4, 115.2, 93.4, 66.3, 45.2, 18.7.

HRMS (ESI) calculated for C₁₂H₁₄ClN₄O: 265.0851 [M+H]⁺, Found: 265.0852.

### Example 3

### Step (c)

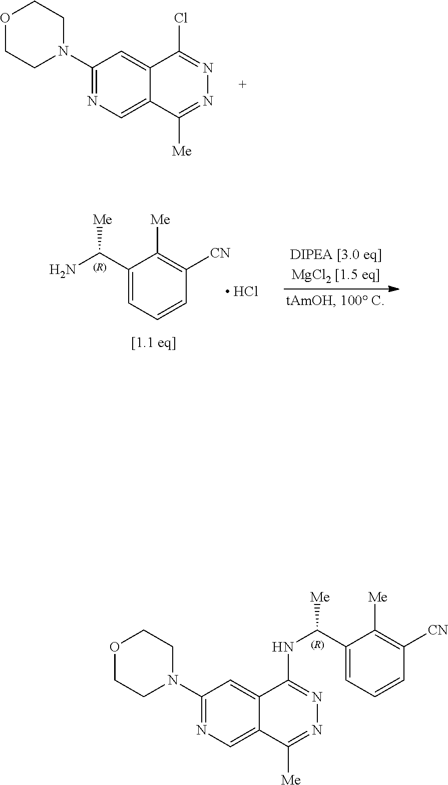

To a 100 L reactor was added 4-(1-chloro-4-methylpyrido[3,4-d]pyridazin-7-yl) morpholine [6.12 kg, 23.1 mol, 1.0 equiv.] and tAmOH [61.2 L]. Then (R)-3-(1-aminoethyl)-2-methylbenzonitrile hydrochloride [5.02 kg, 25.5 mol, 1.1 equiv.] was added followed by MgCl₂ [3.30 kg, 34.7 mol, 1.5 equiv.] and DIPEA [9.04 kg, 69.9 mol, 3.0 equiv.]. At the end of the addition the temperature was increased to 75° C. for 1 h. The reaction was then heated to 100° C. and allowed to react until the starting material was ≤3.0 area % by HPLC analysis (typically 36-48 h). The reaction mixture was cooled to 25° C. and ethyl acetate [55.2 L] was added to the reaction mixture. Water [61.2 L] was added to the mixture at the controlled temperature 20 to 30° C. The aqueous phase was discarded, and volatiles were removed under reduced pressure. Acetonitrile [36.7 L] were added to the obtained mixture and the precipitate was filtered. The filtrate was concentrated under reduced pressure. Acetonitrile [18.4 L] was added and the mixture was heated to 50° C. Upon addition of water, seeding and cooling to 20° C., crystallization was observed. The precipitated solid was filtered, washed once with a pre-mixed solution of water [6.1 L] and acetonitrile [3.0 L]. The wet cake was then dried at 50° C. under vacuum to afford 7.85 kg of (R)-2-methyl-3-(1-((4-methyl-7-morpholinopyrido[3,4-d]pyridazin-1-yl)amino)ethyl)benzonitrile dihydrate in 78% yield.

M.p.: 122.3-122.4° C.

¹H NMR (400 MHz, DMSO-d₆) δ 8.99 (s, 1H), 7.71 (dd, J=7.8, 1.4 Hz, 1H), 7.61 (dd, J=7.8, 1.4 Hz, 1H), 7.53 (d, J=6.8 Hz, 1H), 7.40 (s, 1H), 7.31 (m, 1H), 5.52 (p, J=6.9 Hz, 1H), 3.78 (dd, J=5.8, 3.8 Hz, 4H), 3.68 (dd, J=5.8, 3.8 Hz, 4H), 2.65 (s, 3H), 2.55 (s, 3H), 1.54 (d, J=6.9 Hz, 3H).

¹³C NMR (101 MHz, DMSO-d₆) δ 159.8, 151.2, 149.4, 147.6, 146.4, 139.2, 131.3, 129.7, 127.4, 125.1, 119.0, 114.5, 112.7, 93.6, 66.3, 47.1, 45.5, 21.9, 18.5, 17.2.

HRMS (ESI) calculated for C₂₂H₂₅N₆O:389.2085 [M+H]⁺, Found: 389.2085.

### Example 4

### Step of Making a Fumarate Salt of MRTX0902

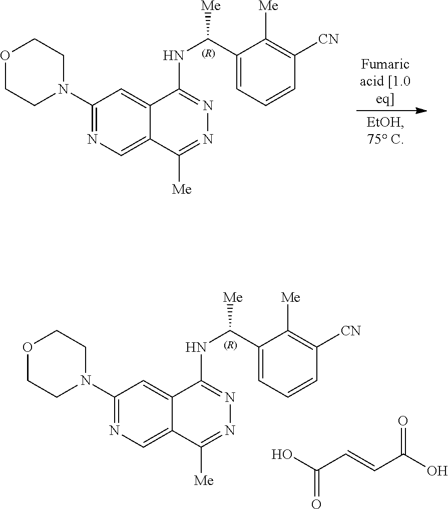

To a 500 L reactor was added (R)-2-methyl-3-(1-((4-methyl-7-morpholinopyrido[3,4-d]pyridazin-1-yl)amino)ethyl)benzonitrile [7.85 kg, 20.2 mol, 1.0 equiv.] and EtOH [86.4 L]. The suspension was heated to 75° C. Crystalline seed material [39.5 g] of the final product was added. Then a prepared solution of fumaric acid [2.40 kg, 20.2 mol, 1.0 equiv.] in 95% aqueous ethanol [52.2 L] was added dropwise. Crystallization was observed upon addition of the fumaric acid solution and stirring was continued at 75° C. for 2 h after the end of the addition. The reaction mixture was cooled to 20° C. over 4 h and then stirring was continued for 4 h at the same temperature. The suspension was filtered and the collected solid was rinsed with EtOH [23.6 L]. The wet cake was then dried at 50° C. under vacuum to afford 8.93 kg of (R)-2-methyl-3-(1-((4-methyl-7-morpholinopyrido[3,4-d]pyridazin-1-yl)amino)ethyl)benzonitrile fumarate in 97% yield.

M.p.: 253.2-253.3° C.

¹H NMR (400 MHz, DMSO-d₆) δ 9.01 (s, 1H), 7.71 (dd, J=7.9, 1.4 Hz, 1H), 7.64-7.55 (m, 2H), 7.42 (s, 1H), 7.32 (t, J=7.8 Hz, 1H), 6.61 (s, 2H), 5.51 (s, 1H), 3.80-3.74 (m, 4H), 3.73-3.66 (m, 4H), 2.65 (s, 3H), 2.56 (s, 3H), 1.54 (d, J=7.0 Hz, 3H).

¹³C NMR (101 MHz, DMSO-d₆) δ 16.7, 17.7, 21.4, 45.0, 46.7, 65.8, 93.2, 112.3, 113.8, 118.5, 124.9, 127.0, 129.2, 130.8, 134.1, 138.7, 145.8, 147.2, 149.3, 150.8, 159.5, 166.2.

HRMS (ESI) calculated for C₂₂H₂₅N₆O:389.2085 [M+H]⁺, Found: 389.2085.

### Example 5

### Step of Making Another Fumarate Salt of MRTX0902

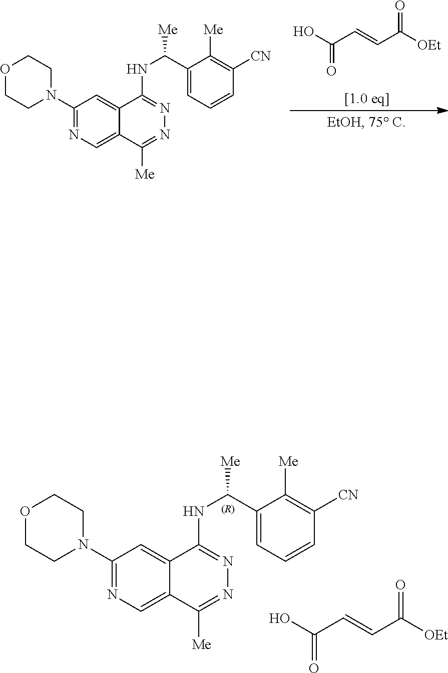

To a 20 mL vial was added (R)-2-methyl-3-(1-((4-methyl-7-morpholinopyrido[3,4-d]pyridazin-1-yl)amino)ethyl)benzonitrile [920 mg, 2.4 mmol, 1.0 equiv.], fumaric acid monoethyl ester [410 mg, 2.8 mmol, 1.2 equiv.] and EtOH [10 mL]. The reaction was heated to 75° C. on a shaker for 1 h. The reaction mixture was then cooled to 20° C. over 15 minutes. The suspension was filtered and the collected solid was dried under vacuum to afford 1.22 g of (R)-2-methyl-3-(1-((4-methyl-7-morpholinopyrido[3,4-d]pyridazin-1-yl)amino)ethyl)benzonitrile (E)-4-ethoxy-4-oxobut-2-enoate in 97% yield.

M.p.: 196.2-196.3° C.

¹H NMR (400 MHz, DMSO-d₆) δ 9.01 (s, 1H), 7.71 (dd, J=7.9, 1.3 Hz, 1H), 7.67-7.58 (m, 2H), 7.41 (s, 1H), 7.32 (t, J=7.8 Hz, 1H), 6.75-6.59 (m, 2H), 5.54-5.48 (m, 1H), 4.19 (q, J=7.1 Hz, 2H), 3.81-3.73 (m, 4H), 3.72-3.64 (m, 4H), 2.65 (s, 3H), 2.56 (s, 3H), 1.54 (d, J=7.1 Hz, 3H), 1.24 (t, J=7.1 Hz, 3H).

¹³C NMR (101 MHz, DMSO-d₆) δ166.3, 165.1, 159.9, 151.3, 149.7, 147.6, 146.3, 139.2, 135.7, 132.6, 131.3, 129.7, 127.4, 125.3, 119.0, 114.4, 112.7, 93.7, 66.3, 61.4, 47.1, 45.5, 21.9, 18.3, 17.2, 14.4.

HRMS (ESI) calculated for C₂₂H₂₅N₆O:389.2085 [M+H]⁺, Found: 389.2085.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A method of manufacturing (R)-2-methyl-3-(1-((4-methyl-7-morpholinopyrido[3,4-d]pyridazin-1-yl)amino)ethyl)benzonitrile, comprising the step of:
a) reacting a compound of the following structure: 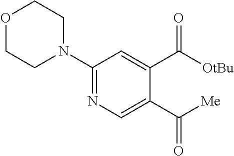 [SMILES: CC(=O)c1cnc(N2CCOCC2)cc1C(=O)OC(C)(C)C]
with hydrazine or a hydrazine salt in the presence of an acid and a solvent to produce a final compound of step a) of the following structure:

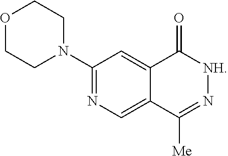 [SMILES: Cc1n[nH]c(=O)c2cc(N3CCOCC3)ncc12]

2. The method of claim 1, wherein the solvent is selected from the group consisting of dimethylacetamide (DMAc), dimethylformamide (DMF), 1,4-dioxane, tetrahydrofuran (THF), 2-methyltetrahydrofuran (2-MeTHF), acetonitrile (MeCN), dimethyl sulfoxide (DMSO), N-methylpyrrolidone (NMP), toluene and an alcohol with a formula R—OH, wherein R is alkyl, allyl or aryl.

3. The method of claim 1, wherein the solvent is ethanol.

4. The method of claim 1, wherein the acid is a mineral acid or an organic acid.

5. The method of claim 4, wherein the acid is a mineral acid.

6. The method of claim 5, wherein the mineral acid is selected from the group consisting of a hydrogen halide of the general formula HX (where X is F, Cl, Br or I), nitric acid, phosphoric acid, sulfuric acid, boric acid and perchloric acid.

7. The method of claim 4, wherein the acid is an organic acid.

8. The method of claim 7, wherein the organic acid is selected from the group consisting of a sulfonic acid of the general formula RSO₃H (where R is alkyl, alkenyl, alkynyl, carbocycle, heterocycle, or aryl), and a carboxylic acid (with one or several carboxylic acid sites) of the general formula RCO₂H (where R is alkyl, alkenyl, alkynyl, carbocycle, heterocycle, or aryl).

9. The method of claim 7, wherein the organic acid is selected from the group consisting of lactic acid, acetic acid, formic acid, citric acid, oxalic acid, uric acid, malic acid, and tartaric acid.

10. The method of claim 1, wherein the acid is acetic acid.

11. The method of claim 1, further comprising step b):
b) reacting the final compound of step a) with a chlorinating agent in the presence of an aprotic solvent to produce a final compound of step b) of the following structure:

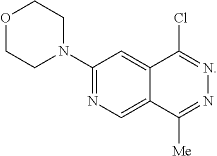 [SMILES: Cc1nnc(Cl)c2cc(N3CCOCC3)ncc12]

12. The method of claim 11, wherein the chlorinating agent is selected from the group consisting of phosphorus oxychloride, phosphorus trichloride, oxalyl chloride, thionyl chloride, diaryl chlorophosphate, dialkyl chlorophosphate, diaryl chlorophosphite, and dialkyl chlorophosphite.

13. The method of claim 11, wherein the chlorinating agent is phosphorus oxychloride.

14. The method of claim 11, wherein the aprotic solvent is selected from the group consisting of chloroform, DMAc, DMF, 1,4-dioxane, THF, 2-MeTHF, MeCN, DMSO, toluene, tertiary amine, and NMP.

15. The method of claim 14, wherein the aprotic solvent is MeCN.

16. The method of claim 11, further comprising step c):
c) reacting the final compound of step b) with a benzylic amine or a benzylic amine salt in the presence of a base, Lewis acid and a high-boiling solvent to produce a final compound of step c) of the following structure:

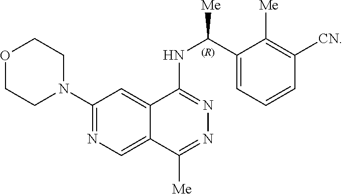

17. The method of claim 16, wherein the base is selected from the group consisting of an organic base and an inorganic base.

18. The method of claim 17, wherein the base is an organic base.

19. The method of claim 18, wherein the organic base is selected from the group consisting of DIPEA, Et₃N, DABCO, and DBU.

20. The method of claim 19, wherein the organic base is DIPEA.

21. The method of claim 17, wherein the base is an inorganic base.

22. The method of claim 21, wherein the inorganic base is selected from the group consisting of carbonate, bicarbonate, and phosphate.

23. The method of claim 16, wherein the Lewis acid is selected from the group consisting of a magnesium salt, a calcium salt, an aluminum-based reagent, and a boron-based reagent.

24. The method of claim 23, wherein the Lewis acid is magnesium chloride.

25. The method of claim 16, wherein the high-boiling solvent is selected from the group consisting of toluene, DMAc, DMF, 1,4-dioxane, DMSO, NMP, and an alcohol with a formula R—OH, wherein R is alkyl, allyl or aryl.

26. The method of claim 25, wherein the high-boiling solvent is tAmOH.

27. A method of manufacturing (R)-2-methyl-3-(1-((4-methyl-7-morpholinopyrido[3,4-d]pyridazin-1-yl)amino)ethyl)benzonitrile, comprising reacting a compound of the following structure 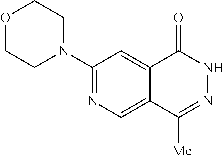 [SMILES: Cc1n[nH]c(=O)c2cc(N3CCOCC3)ncc12]
with a chlorinating agent in the presence of an aprotic solvent to produce a compound of the following structure:

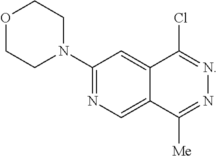 [SMILES: Cc1nnc(Cl)c2cc(N3CCOCC3)ncc12]

28. The method of claim 27, wherein the chlorinating agent is selected from the group consisting of phosphorus oxychloride, phosphorus trichloride, oxalyl chloride, thionyl chloride, diaryl or dialkyl phosphate, diaryl or dialkyl phosphite.

29. The method of claim 27, wherein the chlorinating agent is phosphorus oxychloride.

30. The method of claim 27, wherein the aprotic solvent is selected from the group consisting of chloroform, DMAc, DMF, 1,4-dioxane, THF, 2-MeTHF, MeCN, DMSO, toluene, tertiary amine, and NMP.

31. The method of claim 30, wherein the aprotic solvent is MeCN.

32. A method of manufacturing (R)-2-methyl-3-(1-((4-methyl-7-morpholinopyrido[3,4-d]pyridazin-1-yl)amino)ethyl)benzonitrile, comprising
reacting 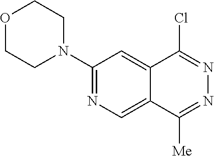 [SMILES: Cc1nnc(Cl)c2cc(N3CCOCC3)ncc12]
with a benzylic amine or a benzylic amine salt in the presence of a base, Lewis acid and a high-boiling solvent to produce a compound with the following structure:

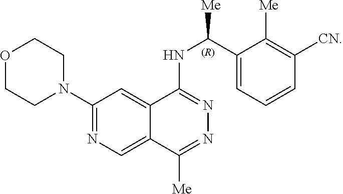

33. The method of claim 32, wherein the base is selected from the group consisting of an organic base and an inorganic base.

34. The method of claim 33, wherein the base is an organic base.

35. The method of claim 34, wherein the organic base is selected from the group consisting of DIPEA, Et₃N, DABCO, and DBU.

36. The method of claim 35, wherein the organic base is DIPEA.

37. The method of claim 33, wherein the base is an inorganic base.

38. The method of claim 37, wherein the inorganic base is selected from the group consisting of carbonate, bicarbonate, and phosphate.

39. The method of claim 32, wherein the Lewis acid is selected from the group consisting of a magnesium salt, a calcium salt, an aluminum-based reagent, and a boron-based reagent.

40. The method of claim 39, wherein the Lewis acid is magnesium chloride.

41. The method of claim 32, wherein the high-boiling solvent is selected from the group consisting of toluene, DMAc, DMF, 1,4-dioxane, DMSO, NMP, and an alcohol with a formula R—OH, wherein R is alkyl, allyl or aryl.

42. The method of claim 41, wherein the high-boiling solvent is tAmOH.

43. A method of manufacturing (R)-2-methyl-3-(1-((4-methyl-7-morpholinopyrido[3,4-d]pyridazin-1-yl)amino)ethyl)benzonitrile, comprising
reacting a compound of the following structure 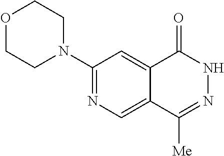 [SMILES: Cc1n[nH]c(=O)c2cc(N3CCOCC3)ncc12]
with a chlorinating agent in the presence of an aprotic solvent to produce a compound of the following structure: 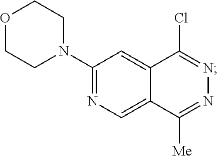 [SMILES: Cc1nnc(Cl)c2cc(N3CCOCC3)ncc12]
reacting 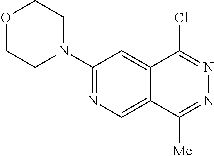 [SMILES: Cc1nnc(Cl)c2cc(N3CCOCC3)ncc12]
with a benzylic amine or a benzylic amine salt in the presence of a base, Lewis acid and a high-boiling solvent to produce a compound with the following structure:

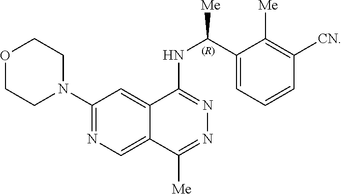

44. A method of manufacturing (R)-2-methyl-3-(1-((4-methyl-7-morpholinopyrido[3,4-d]pyridazin-1-yl)amino)ethyl)benzonitrile, comprising
reacting a compound of the following structure: 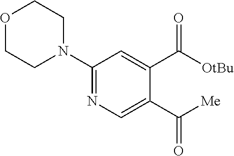 [SMILES: CC(=O)c1cnc(N2CCOCC2)cc1C(=O)OC(C)(C)C]
with hydrazine hydrate in the presence of acetic acid and ethanol to produce a compound with the following structure: 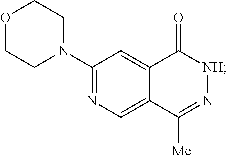 [SMILES: Cc1n[nH]c(=O)c2cc(N3CCOCC3)ncc12]
reacting 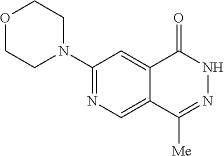 [SMILES: Cc1n[nH]c(=O)c2cc(N3CCOCC3)ncc12]
with phosphorus oxychloride in the presence of MeCN to produce a compound of the following structure: 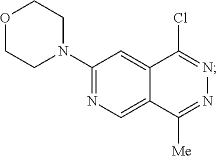 [SMILES: Cc1nnc(Cl)c2cc(N3CCOCC3)ncc12]
reacting 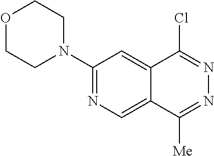 [SMILES: Cc1nnc(Cl)c2cc(N3CCOCC3)ncc12]
with 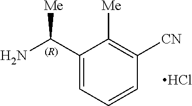
in the presence of DIPEA, MgCl₂, and tAmOH to produce a compound with the following structure:

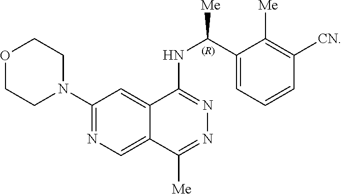

45. A method of manufacturing a fumarate salt of (R)-2-methyl-3-(1-((4-methyl-7-morpholinopyrido[3,4-d]pyridazin-1-yl)amino)ethyl)benzonitrile, comprising
reacting a compound of the following structure: 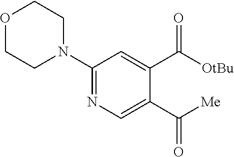 [SMILES: CC(=O)c1cnc(N2CCOCC2)cc1C(=O)OC(C)(C)C]
with hydrazine or a hydrazine salt in the presence of an acid and a solvent to produce a compound with the following structure: 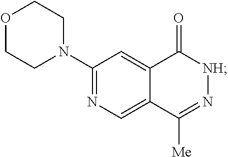 [SMILES: Cc1n[nH]c(=O)c2cc(N3CCOCC3)ncc12]
reacting 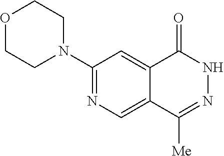 [SMILES: Cc1n[nH]c(=O)c2cc(N3CCOCC3)ncc12]
with a chlorinating agent in the presence of an aprotic solvent to produce a compound of the following structure: 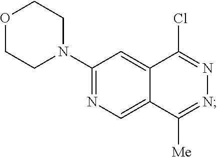 [SMILES: Cc1nnc(Cl)c2cc(N3CCOCC3)ncc12]
reacting 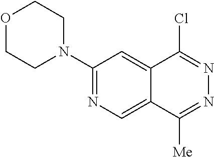 [SMILES: Cc1nnc(Cl)c2cc(N3CCOCC3)ncc12]
with a benzylic amine or a benzylic amine salt in the presence of a base, Lewis acid and a high-boiling solvent to produce a compound with the following structure: 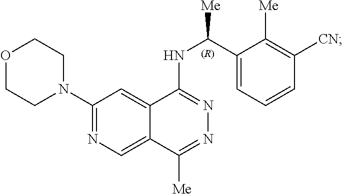
and
reacting 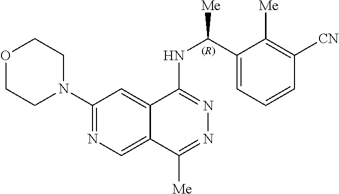
with fumaric acid in the presence of a solvent to produce a compound with the following structure:

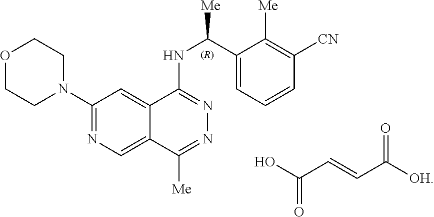

46. A method of manufacturing a fumarate salt of (R)-2-methyl-3-(1-((4-methyl-7-morpholinopyrido[3,4-d]pyridazin-1-yl)amino)ethyl)benzonitrile comprising reacting a compound with the following structure: 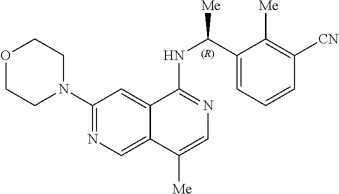
with fumaric acid in the presence of a solvent to produce a compound with the following structure:

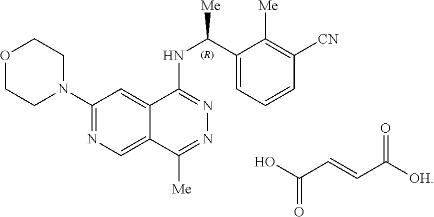

47. The method of claim 46, wherein the solvent is selected from the group consisting of DMAc, DMF, THF, 2-MeTHF, MeCN, DMSO, NMP, toluene and an alcohol with a formula R—OH, wherein R is alkyl, allyl or aryl.

48. The method of claim 46, wherein the solvent is ethanol.

49. A method of manufacturing a fumarate salt of (R)-2-methyl-3-(1-((4-methyl-7-morpholinopyrido[3,4-d]pyridazin-1-yl)amino)ethyl)benzonitrile, comprising
reacting a compound of the following structure: 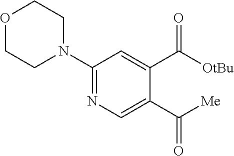 [SMILES: CC(=O)c1cnc(N2CCOCC2)cc1C(=O)OC(C)(C)C]
with hydrazine hydrate in the presence of acetic acid and ethanol to produce a compound with the following structure: 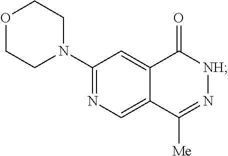 [SMILES: Cc1n[nH]c(=O)c2cc(N3CCOCC3)ncc12]
reacting 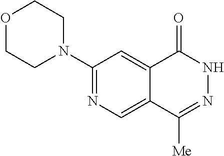 [SMILES: Cc1n[nH]c(=O)c2cc(N3CCOCC3)ncc12]
with phosphorus oxychloride in the presence of MeCN to produce a compound of the following structure: 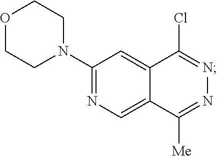 [SMILES: Cc1nnc(Cl)c2cc(N3CCOCC3)ncc12]
reacting 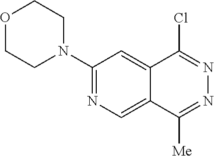 [SMILES: Cc1nnc(Cl)c2cc(N3CCOCC3)ncc12]
with 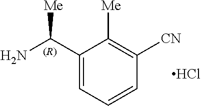
in the presence of DIPEA, MgCl₂, and tAmOH to produce a compound with the following structure: 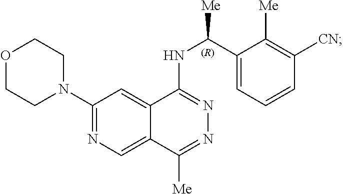
and
reacting 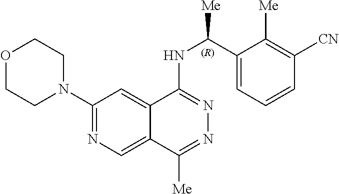
with fumaric acid in the presence of ethanol to produce a compound with the following structure:

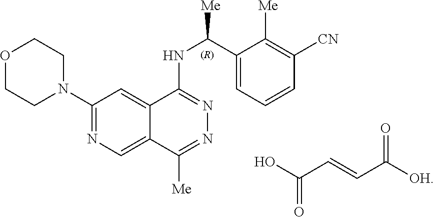

50. A compound selected from the group consisting of:

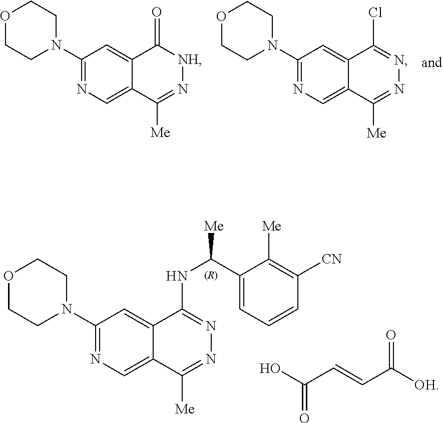

51. A compound of the following structure: [chemical structure]
